(19) United States
(12) Patent Application Publication
(10) Pub. No.: US 2003/0095979 A1
(43) Pub. Date: May 22, 2003

(54) Pharmaceutical preparations comprising modified peptides

(75) Inventors: Frank Mattner, Vienna (AT); Wolfgang Zauner, Vienna (AT); Walter Schmidt, Vienna (AT); Michael Buschle, Perchtoldsdorf (AT)

Correspondence Address: Mark B. Wilson, Fulbright & Jaworski L.L.P., 600 Congress Avenue, Suite 2400, Austin, TX, 78701, US

(73) Assignee: Cistem Biotechnologies GMBH, Vienna (AT)

(21) Appl. No.: 10/269,171

(22) Filed: Oct. 11, 2002

Related U.S. Application Data
(63) Continuation of application No. PCT/EP01/04313, filed on Apr. 17, 2001.

(51) Int. Cl. A61K 48/00; A61K 38/16; A61K 39/00

(52) U.S. Cl. 424/185.1; 514/8; 514/12

(57) ABSTRACT

Pharmaceutical preparation comprising a peptide of the formula

X_{N}-Peptide_{L}-X_{M},

wherein X is an amino acid residue selected from Gly, Ala, Val, Leu, Ile, Pro, Phe, Tyr, Trp, Cys, Met, Glu and Asp; Peptide_{L} is a potentially immunogenic fragment consisting of L amino acid residues; L is an integer from 6 to 100, N and M are integers from 0 to 2L, with the proviso that either N or M is at least 2; X_{N} and X_{M} being amino acid sequences not occurring at this position and in this constellation with Peptide_{L} and a polycationic substance.

The invention relates to pharmaceutical preparations comprising modified peptide antigens, especially peptides suitable for vaccination.

Peptides become increasingly important in modern vaccine design. It has been shown that co-injection of a mixture of poly-L-arginine or poly-L-lysine together with an appropriate peptide as a vaccine protect animals from tumor growth in mouse models (Buschle et al., Gene Ther Mol Biol 1 (1998) 309-321); Schmidt et al., PNAS 94 (1997), 3262-3267). This chemically defined vaccine is able to induce high numbers of antigen/peptide-specific T cells. In order to induce antigen-specific T cells, peptides need to be taken up by antigen presenting cells (APC). These APCs induce an immune cascade eventually leading to the induction of antigen-specific immune effector cells, for example cytotoxic T cells. Antigenic peptides have to form a “depot” allowing APCs sufficient time to infiltrate the (vaccine) injection site. Unfortunately, most peptides fail to form such a depot, even when injected together with an adjuvant. It is a well recognized problem in the art that many promising peptides defining antigenic regions of medically important pathogens fail to provide a sufficient immune response in vivo.

WO 97/40754 relates to a method of improving the binding affinity of a peptide epitope for MHC class II molecules by attaching to the N-terminus of the peptide epitope a hydrophobic amino acid sequence. This modified peptide shows an improved capability of binding to MHC class II molecules whereby these so formed complexes are used to inactivate T cells bearing receptors that recognize an epitope on the modified peptide. The formed complexes can therefore be used therapeutically for inactivating unwanted immune responses, e.g. autoimmune reponses. Therefore the aim of the modified peptides according to the WO 97/40754 is not an enhanced uptake in antigen presenting cells, but to improve the binding of the peptide epitope to MHC class II molecules and therefore to inactivate T cells. This is contrary to the activation of T cells by antigen presenting cells which take up the antigen, process it and display the processed fragments of the antigen on their cell surface mediated by MHC molecules.

In U.S. Pat. No. 5,726,292 a construct is described comprising a protein, protein fragment, polypeptide or peptide, a hydrophobic anchor and a proteosome, which construct is used as an immunogenic composition for the use as therapeutic agent and vaccine. This construct shows improved immuno potentiating activity. The hydrophobic anchor may be a hydrophobic peptide of about 3 to 50 amino acids in length. It is shown in this document that the peptide comprising the hydrophobic amino acid sequence alone (without a proteosome) was not immunogenic, whereas the same peptide comprising the hydrophobic tail complexed to proteosomes showed high immunogenic activities due to the anchoring in the lipid component.

It is therefore an object of the present invention to provide pharmaceutical preparations comprising peptides which induce a sufficient immune response in a mammal, especially in humans, to which this peptide is applied. It is a further object to provide compositions comprising modified peptides derived from wild type antigens which allow a stronger immune response than the wild type peptide. Further, according to the present invention non-immunogenic peptides being antigens shall be modified to become immunogenic. Yet another object is to improve the immunogenic quality of peptide antigens in pharmaceutical compositions.

These objects are solved by a pharmaceutical preparation comprising peptides of the formula

X_{N}-Peptide_{L}-X_{M},

wherein x is an amino acid residue selected from Gly, Ala, Val, Leu, Ile, Pro, Phe, Tyr, Trp, Cys, Met, Glu and Asp; Peptide_{L} is a potentially immunogenic fragment consisting of L amino acid residues, which may be derived from a naturally occurring protein; alternatively, PeptideL may also be a synthetically designed immunogenic peptide; L is an integer from 6 to 100, N and M are integers from 0 to 2L, with the proviso that either N or M is at least 2; X_{N} and X_{M} being amino acid sequences not occurring at this position and in this constellation with Peptide_{L} (i.e. not naturally occuring, if derived from a naturally occuring substance and not described at this position/constellation, if derived from a synthetic peptide design method), and a polycationic substance.

The present inventors have observed that especially hydrophobic peptides are able to induce specific T cells, e.g. the tyrosinase-related protein-2-derived peptide VYDFFVWL. However, many peptides which are antigens, potentially suitable as a vaccine, are not very hydrophobic per se. Therefore, the adding of amino acids according to the present invention at the N- and/or C-terminus to a known (non-hydrophobic) peptide sequence in combination with a polycationic substance renders such a peptide hydrophobic and therefore more immunogenic. With the present invention it could be shown that the more hydrophobic amino acids were added at the N- and/or the C-terminus of a neutral or hydrophilic antigen, the more peptide specific T cells were induced by such a modified antigen, if these modified peptides are combined with polycationic substances.

In principle any peptide can be modified to become an immunogenic peptide in the composition according to the present invention. It is, however, preferred to modify peptides encoding for antigenic determinants according to the present invention. Especially peptides with a low hydrophobicity or even hydrophilic peptides which—although perhaps coding for an exposed surface determinant of a pathogen—elicit no effective immune response (i.e. are only potentially pathogenic), may be transformed to efficient immunogenics if modified according to the present invention. It is preferred to provide for Leu, Ile, Phe, Trp, Cys to form hydrophobic tails to the given peptide. If the PeptideL is a peptide derived from natural sources by fragmentation of pathogen proteins, the length should be selected suitable to sterically define e.g. the antigenic determinant. Preferably, L is an integer from 8 to 25.

Preferably, the amino acid residues are connected via peptide bonds with PeptideL and with each other. Such peptides may easily and economically be produced by recombinant expression or Merrifield solid phase techniqes. These methods lead to peptide-bond linked amino acid chains. However, other covalent chemical connections between the single amino acid residues are also possible, especially disulfide bonds (C-C) or other connections wherein functional groups of the amino acid residues are affected. In the present specification (if not otherwise indicated) amino acid residues are linked via peptide bonds (i.e. “CC” means C bond via peptide bond to C; “C—C” means C bound via disulfide bound to C).

The length of the “tail” added to the PeptideL is preferably 2 to 2L amino acid residues and located at the C- or at the N-terminus or at both termini.

More preferred lengths of the peptide tails added to the “wild type” peptide antigen are from 2 to 10 amino acid residues, especially if Peptide_{L} has from 6 to 15 amino acid residues.

According to a preferred embodiment of the present invention, the peptide tail contains at least 50% amino acid residues selected from Phe, Leu, Ile, Trp and Cys. Especially preferred are peptides wherein X_{N} and/or X_{M} contain at least 30% Phe residues.

Preferably, the hydrophobic peptide tail according to the present invention has a cumulative hydrophobicity of more than 1.3, especially 1.5 per amino acid residue according to the calculations of Rao et al. (Biochimica et Biophysica Acta 869 (1986), 197-214). According to another preferred embodiment, the hydrophobic peptide tail has a total cumulative hydrophobicity (Rao et al.) of more than 8 for 6 amino acids. For longer or shorter sequences, similar minimum cumulative hydrophobicity scores (Rao et al.) are preferred.

On the other hand, it could also be shown that the addition of negatively charged amino acids at the N- and/or the C-terminus of a known peptide also leads to improved immunogenic properties, to a “depot” formation. According to another preferred embodiment of the present invention XN and/or XM contain at least 30% of residues selected from Asp and Glu.

According to the present invention it is therefore preferred that X is either selected from Gly, Ala, Val, Leu, Ile, Pro, Phe, Trp, Cys, especially Leu, Ile, Phe, Trp, Cys or from the group Cys, Glu and Asp, especially Glu and Asp in order to form either a hydrophobic tail or a negatively charged tail.

Therefore, preferred peptides to be used in the composition according to the present invention have XN and/or XM selected from (FI)₁₋₅, (FI)₁₋₅W, (FI)₁₋₅W(FI)₁₋₅, (FL)₁₋₅ (FL)₁₋₅W, (FL)₁₋₅W(FL)₁₋₅, (FI)₁₋₅ (FL)₁₋₅, (FL)₁₋₅(FI)₁₋₅, F(II)₁₋₅, (FIL)₁₋₅, (FLI)₁₋₅, (FLL)₁₋₅, (FIF)₁₋₅, (FLF)₁₋₅, (WIL)₁₋₅ (WLI)₁₋₅, (LWI)₁₋₅, (IWL)₁₋₅, (WI)₁₋₅, (WL)₁₋₅, (WL)₁₋₅F, (WI)₁₋₅F, (FV)₁₋₅, (FVI)₁₋₅, (FAI)₁₋₅ (FIA)₁₋₅, (FIV)₁₋₅, (FAI)₁₋₅, (FV)₁₋₅C—C(FV)₁₋₅, (FA)₁₋₅C—C(FA)₁₋₅ and (FV)₁₋₅C—C(FA)₁₋₅, (FI)₁₋₅C—C(FI)₁₋₅, (FL)₁₋₅C—C(FL)₁₋₅, (WI)₁₋₅C—C(WI)₁₋₅; (FI)₁₋₅WC—C, (FI)₁₋₅C—C, (FL)₁₋₅WC—C, (FL)₁₋₅C—C, (WI)₁₋₅C—C, (WI)₁₋₅FC—C, wherein F is Phe, I is Ile, W is Trp, C is Cys, C-C is a cystein bridge, L is Leu, V is Val and A is Ala.

Preferred peptides with negatively charged “tails” include peptides, wherein X_{N} and/or X_{M} is selected from (ED)₁₋₅, (EDE)₁₋₅, (DED)1-5, (DE)₁₋₅, (DEE)₁₋₅, (EED)₁₋₅, (EXE)₁₋₅, (DXD)₁₋₅, (EXD)₁₋₅, (EX)₁₋₅, (DX)₁₋₅, (ED)₁₋₅(FI)₁₋₅ and (FI)₁₋₅(ED)₁₋₅, (ED)₁₋₅C—C(ED)₁₋₅, (ED)₁₋₅C—C, wherein X is selected from Gly, Ala, Val, Leu, Ile, Pro, Phe, Tyr, Trp, Cys and Met and E is Glu, D is Asp, F is Phe, I is Ile, C is Cys and C-C is a cystein bridge.

The present pharmaceutical preparations are especially suitable for vaccination. Therefore, the present invention is also drawn to a vaccine comprising a pharmaceutical preparation according to the present invention, optionally with further pharmaceutically acceptable carrier substances, especially adjuvants. Primary adjuvants in the composition according to the present invention are, of course, polycationic substances which are especially suited when negatively charged amino acid “tails” are used as X_{N} and/or X_{M}.

The polycationic compound(s) to be used according to the present invention may be any polycationic compound which shows the characteristic effects according to the WO 97/30721. Preferred polycationic compounds are selected from basic polypeptides, organic polycations, basic pdlyamino acids or mixtures thereof. These polyamino acids should have a chain length of at least 4 amino acid residues (see: Tuftsin as described in Goldman et al. (1983)). Especially preferred are substances like polylysine, polyarginine and polypeptides containing more than 20%, especially more than 50% of basic amino acids in a range of more than 8, especially more than 20, amino acid residues or mixtures thereof. Other preferred polycations and their pharmaceutical compositons are described in WO 97/30721 (e.g. polyethyleneimine) and WO 99/38528. Preferably these polypeptides contain between 20 and 500 amino acid residues, especially between 30 and 200 residues.

These polycationic compounds may be produced chemically or recombinantly or may be derived from natural sources.

Cationic (poly)peptides may also be anti-microbial with properties as reviewed in Ganz et al, 1999; Hancock, 1999. These (poly)peptides may be of prokaryotic or animal or plant origin or may be produced chemically or recombinantly (Andreu et al., 1998; Ganz et al., 1999; Simmaco et al., 1998). Peptides may also belong to the class of defensins (Ganz, 1999; Ganz et al., 1999). Sequences of such peptides can be, for example, be found in the Antimicrobial Sequences Database under the following internet address:

http://www.bbcm.univ.trieste.it/˜tossi/naal.html

Such host defence peptides or defensives are also a preferred form of the polycationic polymer according to the present invention. Generally, a compound allowing as an end product activation (or down-regulation) of the adaptive immune system, preferably mediated by APCs (including dendritic cells) is used as polycationic polymer.

Especially preferred for use as polycationic substance in the present invention are cathelicidin derived antimicrobial peptides or derivatives thereof (Austrian patent application A 1416/2000, incorporated herein by reference), especially antimicrobial peptides derived from mammal cathelicidin, preferably from human, bovine or mouse.

Polycationic compounds derived from natural sources include HIV-REV or HIV-TAT (derived cationic peptides, antennapedia peptides, chitosan or other derivatives of chitin) or other peptides derived from these peptides or proteins by biochemical or recombinant production. Other preferred polycationic compounds are cathelin or related or derived substances from cathelin. For example, mouse cathelin is a peptide which has the amino acid sequence NH₂-RLAGLLRKGGEKIGEKLKKIGOKIKNFFQKLVPQPE-COOH. Related or derived cathelin substances contain the whole or parts of the cathelin sequence with at least 15-20 amino acid residues. Derivations may include the substitution or modification of the natural amino acids by amino acids which are not among the 20 standard amino acids. Moreover, further cationic residues may be introduced into such cathelin molecules. These cathelin molecules are preferred to be combined with the antigen. These cathelin molecules surprisingly have turned out to be also effective as an adjuvant for a antigen without the addition of further adjuvants. It is therefore possible to use such cathelin molecules as efficient adjuvants in vaccine formulations with or without further immunactivating substances.

Another preferred polycationic substance to be used according to the present invention is a synthetic peptide containing at least 2 KLK-motifs separated by a linker of 3 to 7 hydrophobic amino acids (Austrian patent application A 1789/2000, incorporated herein by reference).

Immunostimulatory deoxynucleotides are e.g. natural or artificial CpG containing DNA, short stretches of DNA derived from non-vertebrates or in form of short oligonucleotides (ODNs) containing non-methylated cytosine-guanine di-nucleotides (CpG) in a certain base context (e.g. Krieg et al., 1995) but also inosine containing ODNs (I-ODNs) as described in the AT patent application A 1973/2000 or poly I:C and oligo dI:C as described in A 1000/2000.

Neuroactive compounds, e.g. combined with polycationic substances are described in PCT/EP00/09657.

The vaccine compositions according to the present invention may be formulated according to known methods, e.g. as i.v. vaccines, DNA vaccines, oral vaccines, transdermal vaccines, topical vaccines, intranasal vaccines and as combination vaccines. The dosages may be selected by standard processes. For vaccines which are improvements of known vaccines, however, a lower dosage than the known vaccine is possible for the same protection and therefore preferred.

Preferably, the vaccine is provided in a storage stable form, e.g. lyophilized, optionally provided in combination with a suitable reconstitution solution.

The invention will be described in more detail by the following examples and the drawing figures, but would of course not be restricted thereto.

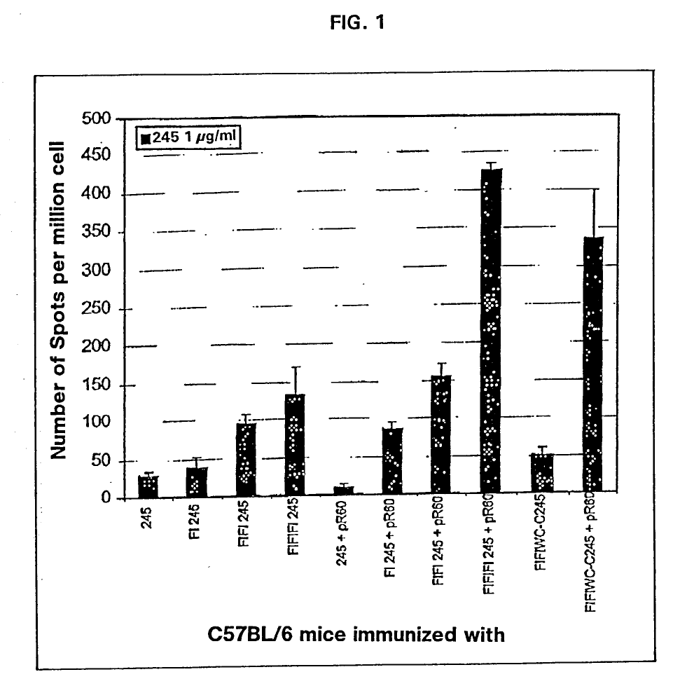

FIG. 1 shows the immunization with an ovalbumin derived peptide with a hydrophobic tail and (optionally) polyarginine.

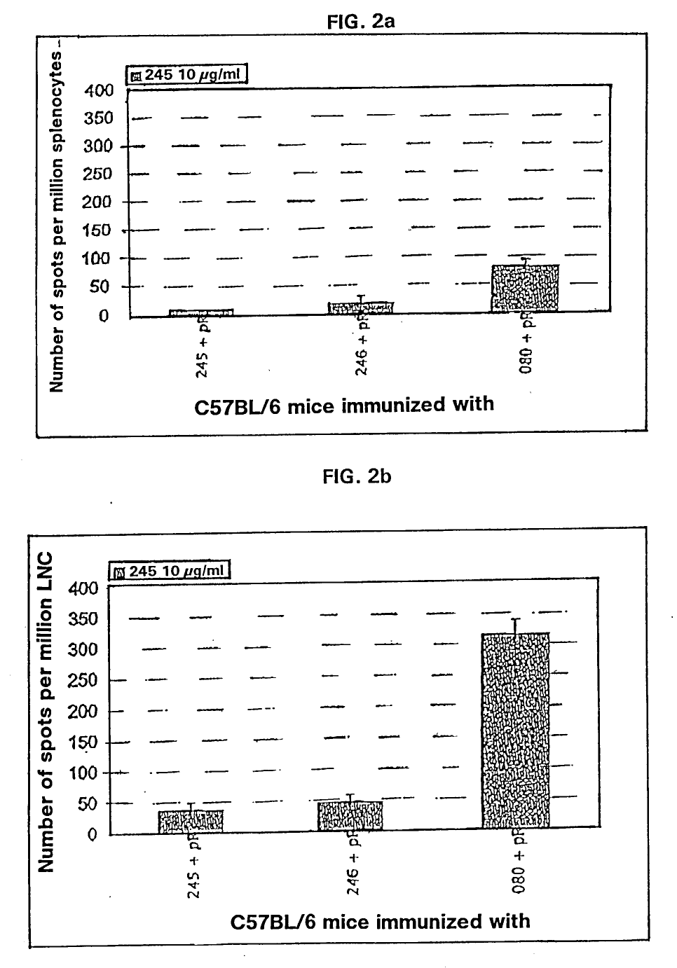

FIG. 2 shows the immunization with an ovalbumin derived peptide and a negatively charged amino acid tail with polyarginine.

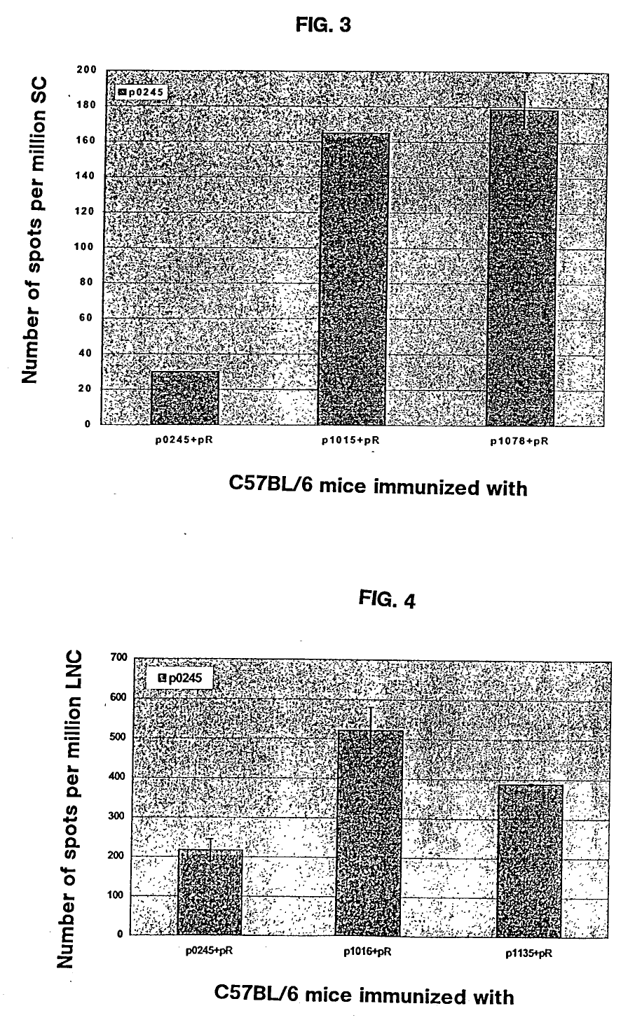

FIG. 3 shows the immunization with an ovalbumin derived, class I H-2 Kb-restricted peptide which was made “more hydrophobic” by directly adding F and I at the C-terminus.

FIG. 4 shows the immunization with an ovalbumin peptide which was made “more hydrophobic” by directly adding L and A at the N-terminus.

### EXAMPLE 1

The ovalbumin derived class I H-2K^{b}, restricted peptide (“245”), SIINFEKL (which is a weak inducer of T cells, alone or in combination with an adjuvant like poly-L-arginine) was made “more hydrophobic by directly adding F and I or by adding FIFIW via a C—C bridge. Groups of 4 mice (C75BL/6, femal, 8 weeks of age, H-2^{b} were injected subcutaneoulsy in the flank 3 times (days 0, 21 and 28) as follows (dose 100 μg of peptide, molarity adapted; +/−100 μg poly-L-arginine per mouse):

1) SIINFEKL (peptide “245”, 4 of 8 amino acids (aa) hydrophobic)

2) FISIINFEKL (6 of 10 aa hydrophobic)

3) FIFISIINFEKL (8 of 12 aa hydrophobic)

4) FIFIFISIINFEKL (10 of 14 aa hydrophobic)

5) SIINFEKL+poly-L-arginine

6) FISIINFEKL+poly-L-arginine

7) FIFISIINFEKL+poly-L-arginine

8) FIFIFISIINFEKL+poly-L-arginine

9) FIFIWC-CSIINFEKL (11 of 15 aa hydrophobic)

10) FIFIWC-CSIINFEKL+poly-L-arginine

One week after the third vaccination spleens were removed and the splenocytes were activated ex vivo with peptide SIINFEKL (“245”) to determine IFN-y-producing specific cells (ELISpot assay).

The more hydrophobic amino acids were added in the N-terminus of peptide SIINFEKL, the more peptide specific T cells were induced (see FIG. 1). Peptides FIFISIINFEKL and FIFIFISIINFEKL were even able to induce about 100 peptide specific T cells per 1 million spleen cells without adding poly-L-arginine. Peptide FIFIFISIINFEKL in combination with poly-L-arginine induced more than 400 SIINFEKL specific T cells among 1 million splenocytes (similar to peptide FIFIWC-CSIINFEKL).

### EXAMPLE 2

The neutral peptide SIINFEKL (one negatively charged (E), one positively charged (K) amino acid) was rendered negative by adding (at the N-terminus) EE or EDED, respectively.

Groups of 4 mice (C57BL/6, female, 6 weeks of age, II-2^{b}) were injected subcutaneously in the footpad 3 times (days 0, 14, 28) as follows (dose 100 ug of peptide, molarity adapted; +100 μg poly-L-arginine per mouse):

1) SIINFEKL (peptide “1245”)+poly-L-arginine

2) EESIINFEKL (peptide “246”)+poly-L-arginine

3) EDEDSIINFEKL (peptide “080”)+poly-L-arginine

Groups of 4 mice were vaccinated subcutaneously in the footpad 3 times as indicated. One week after the 3rd vaccination, popliteal lymph nodes and spleens were removed and lymph node cells and splenocytes were activated ex vivo with peptide SIINFEKL (“245”) to determine IFN-y-producing specific cells (ELISpot assay).

As can be seen in FIG. 2, the addition of 4 negatively-charged amino acids (EDED) at the N-terminus of peptide SIINFEKL makes this peptide (in combination with poly-L-arginine) able to induce a high amount of specific IFN-γ-producing T cells in the draining (popliteal) lymph node (local response) and in the spleen (systemic response).

Thus, the addition of hydrophobic amino acids as well as the addition of negatively charged amino acids transforms the peptide SIINFEKL to a good inducer of specific T cells.

### EXAMPLE 3

C-terminus Instead of N-terminus

SIINFEKL with 4 hydrophobic (bold) amino acids was made “more hydrophobic” by directly adding F and I at the C-terminus. The already described (N-terminus-prolonged) peptide FIFISIINFEKL served as control for SIINFEKLFIFI.

Groups of 4 mice (C57BL/6, female, 8 weeks of age, H-2^{b}) were injected subcutaneously into the hind footpads as follows (dose 100 μg of peptide, molarity adapted; +100 μg poly-L-arginine per mouse):

1) SIINFEKL 8peptide “245”)+poly-L-arginine (4 of 8 aa hydrophobic)

2) FIFISIINFEKL (peptide “1015”)+poly-L-arginine (8 of 12)

3) SIINFEKLFIFI (peptide “1078”)+poly-L-arginine (8 of 12)

Groups of 4 mice were vaccinated subcutaneously in the hind footpads as indicated. 7 days after the vaccination single cell suspensions of spleens were prepared and cells were restimulated ex vivo to detect IFN-γ-producing cells via ELISpot assay.

As can be seen in FIG. 3 in contrast to peptide SIINFEKL, the peptides FIFISIIFEKL and SIINFEKLFIFI (in combination with poly-L-arginine) were able to induce high numbers of SIINFEKL-specific IFN-γ-producing T cells.

Thus, the addition of hydrophobic amino acids works equally well (with regard to induction of peptide-specific T cells) at the N-terminus or C-terminus, respectively.

### EXAMPLE 4

L and A Instead of F and I

The peptide SIINFEKL with 4 hydrophobic (bold) amino acids was made “more hydrophobic” by directly adding L and A at the N-terminus. The already described (N-terminus-prolonged) peptide FIFIFISIINFEKL served as control for the peptide LALALASIINFEKL.

Groups of 4 mice (C57BL/6, female, 8 weeks of age, H-2^{b}) were injected subcutaneously into the hind footpads as follows (dose 100 μg of peptide, molarity adapted; +100 μg poly-L-arginine per mouse):

1) SIINFEKL (peptide “245”)+poly-L-arginine

2) FIFIFISIINFEKL (peptide “1016”)+poly-L-arginine

3) LALALASIINFEKL (peptide “1135”)+poly-L-arginine

The result of the vaccination experiment with the peptide SIINFEKL which was made “more hydrophobic” by directly adding L and A at the N-terminus is shown in FIG. 4.

1. Pharmaceutical preparation comprising a peptide of the formula
X_{N}—Peptide_{L}—X_{M′}
wherein X is an amino acid residue selected from Cly, Ala, Val, Leu, Ile, Pro, Phe, Tyr, Trp, Cys, Met, Glu and ASp; Peptide_{L} is a potentially immunogenic fragment consisting of L amino acid residues; L is an integer from 6 to 100, N and M are integers from 0 to 2L, with the proviso that either N or M is at least 2; X_{N} and X_{M} being amino acid sequences not occuring at this position and in this constellation with Peptide_{L} and a ploycationic substance.

2. Pharmaceutical preparation according to claim 1 characterized in that L is an integer from 8 to 25.

3. Pharmaceutical preparation according to claim 1 or 2, characterized in that M is O and N is an integer from 2 to 2L.

4. Pharmaceutical preparation according to any one of claims 1 to 3, characterized in that M or N is an integer from 2 to 10 and L is an integer from 6 to 15.

5. Pharmaceutical preparation according to any one of claims 1 to 4, characterized in that it contains at least 50% amino acid residues selected from Phe, Leu, Ile, Trp and Cys.

6. Pharmaceutical preparation according to any one of claims 1 to 5 characterized in that X_{N} and/or X_{M} contain at least 30% Phe residues.

7. Pharmaceutical preparation according to any one of claims 1 to 5 characterized in that X_{N} and/or X_{M} contain at least 30% residues selected from Asp and Glu.

8. Pharmaceutical preparation according to claim 1 characterized in that X_{N} and/or X_{M} is selected from (FI)₁₋₅, (FI)₁₋₅W, (FI)₁₋₅W(FI)₁₋₅, (FL)₁₋₅, (FL)₁₋₅W, (FL)₁₋₅W(FL)₁₋₅, (FI)₁₋₅(FL)₁₋₅, (FL)₁₋₅(FI)₁₋₅, F(II)₁₋₅, (FIL)₁₋₅, (FLI)₁₋₅, (FLL)₁₋₅, (FIF)₁₋₅, (FLF)₁₋₅, (WIL)₁₋₅, (WLI)₁₋₅, (LWI)₁₋₅, (IWL)₁₋₅, (WI)₁₋₅, (WL)₁₋₅, (WL)₁₋₅F, (WI)₁₋₅F, (FV)₁₋₅(FA)₁₋₅C-C(FA)₁₋₅ and (FV)₁₋₅C-C(FA)₁₋₅, (FI)₁₋₅C-C(FI)₁₋₅, (FL)₁₋₅C-C(FL)₁₋₅, (WI)₁₋₅C-C(WI)₁₋₅, (FI)₁₋₅WC-C, (FI)₁₋₅C-C, (FL)₁₋₅WC-C, (FL)₁₋₅C-C, (WI)₁₋₅C-C, (WI)₁₋₅FC-C, wherein F is Phe, I is Ile, W is Trp, C is Cys, C-C is a cystein bridge, L is Leu, V is Val and A is Ala.

9. Pharmaceutical preparation according to claim 1, characterized in that Xₙ and/or X_{M} is selected from (ED)_{1-5′}, (EDE)₁₋₅, (DED)₁₋₅, (DE)₁₋₅, (DEE)₁₋₅, (EED)₁₋₅, (EXE)₁₋₅, (DXD)₁₋₅, (EXD)₁₋₅, (EX)₁₋₅, (DX)₁₋₅, (ED)₁₋₅(FI)₁₋₅ and (FI)₁₋₅(ED)₁₋₅, (ED)₁₋₅C-C(ED)₁₋₅, (ED)₁₋₅C-C, wherein X is selected from Gly, Ala, Val, Leu, Ile, Pro, Phe, Tyr, Trp, Cys and Met and E is Glu, D is Asp, F is Phe, I is Ile, C is Cys and C-C is a cystein bridge.

10. Pharmaceutical preparation according to claims 1 to 9, characterized in that the polycationic substance is selected from the group consisting of basic polypeptides, organic polycations, polycationic antimicrobial peptides, especially cathalicitin derived antimicrobial peptides, a KLK Hy₃₋₇KLK, wherein Hy₃₋₇ is a linker of 3-7 hydrophobic amino acids, an immungenic oligodeoxynucleotide (ODN), especially ODNs with CpG motifs or inosine containing ODNs, or mixtures thereof.

11. Vaccine comprising a pharmaceutical preparation according to claims 1 to 9.


SEQUENCE LISTING (11 sequences)

SEQ ID NO: 1 - protein, 8 aa, Artificial Sequence
  Description of Artificial Sequence Synthetic Peptide
VYDFFVWL

SEQ ID NO: 2 - protein, 36 aa, Artificial Sequence
  Description of Artificial Sequence Synthetic Peptide
RLAGLLRKGGEKIGEKLKKIGXKIKNFFQKLVPQPE

SEQ ID NO: 3 - protein, 8 aa, Artificial Sequence
  Description of Artificial Sequence Synthetic Peptide
SIINFEKL

SEQ ID NO: 4 - protein, 9 aa, Artificial Sequence
  Description of Artificial Sequence Synthetic Peptide
FISINFEKL

SEQ ID NO: 5 - protein, 12 aa, Artificial Sequence
  Description of Artificial Sequence Synthetic Peptide
FIFISIINFEKL

SEQ ID NO: 6 - protein, 14 aa, Artificial Sequence
  Description of Artificial Sequence Synthetic Peptide
FIFIFISIINFEKL

SEQ ID NO: 7 - protein, 15 aa, Artificial Sequence
  Description of Artificial Sequence Synthetic Peptide
FIFIWCCSIINFEKL

SEQ ID NO: 8 - protein, 10 aa, Artificial Sequence
  Description of Artificial Sequence Synthetic Peptide
EESIINFEKL

SEQ ID NO: 9 - protein, 12 aa, Artificial Sequence
  Description of Artificial Sequence Synthetic Peptide
EDEDSIINFEKL

SEQ ID NO: 10 - protein, 12 aa, Artificial Sequence
  Description of Artificial Sequence Synthetic Peptide
SIINFEKLFIFI

SEQ ID NO: 11 - protein, 14 aa, Artificial Sequence
  Description of Artificial Sequence Synthetic Peptide
LALALASIINFEKL